(19) United States
(12) Patent Application Publication
(10) Pub. No.: US 2014/0018597 A1
(43) Pub. Date: Jan. 16, 2014

(54) PROCESS FOR TREATING AN OUTPUT FROM A HYDROCARBON CONVERSION BY WASHING WITH AN AQUEOUS MEDIUM

(71) Applicant: BASF SE, Ludwigshafen (DE)

(72) Inventors: Daniel Pfeiffer, Neustadt (DE); Stefan Bitterlich, Dirmstein (DE); Michael Hübner, Lampertheim (DE)

(73) Assignee: BASF SE, Ludwigshafen (DE)

(21) Appl. No.: 13/937,413

(22) Filed: Jul. 9, 2013

Related U.S. Application Data
(60) Provisional application No. 61/670,142, filed on Jul. 11, 2012.

(51) Int. Cl. C07C 7/10

(52) U.S. Cl. CPC C07C 7/10
USPC 585/803; 585/868

(57) ABSTRACT

The present invention relates to a process for treating an output from a hydrocarbon conversion, wherein the hydrocarbon conversion is performed in the presence of an acidic ionic liquid. The hydrocarbon conversion is preferably an isomerization. A mixture which originates from the hydrocarbon conversion and comprises at least one hydrocarbon and at least one hydrogen halide is washed with an aqueous medium having a pH between 5 and 9, which removes hydrogen halide from the mixture.

### CROSS-REFERENCE TO RELATED APPLICATIONS

This patent application claims the benefit of pending U.S. provisional patent application Ser. No. 61/670,142 filed on Jul. 11, 2012, incorporated in its entirety herein by reference.

The present invention relates to a process for treating an output from a hydrocarbon conversion, wherein the hydrocarbon conversion is performed in the presence of an acidic ionic liquid. The hydrocarbon conversion is preferably an isomerization. A mixture which originates from the hydrocarbon conversion and comprises at least one hydrocarbon and at least one hydrogen halide is washed with an aqueous medium having a pH between 5 and 9, which removes hydrogen halide from the mixture.

Ionic liquids can be used in various hydrocarbon conversion processes; they are especially suitable as catalysts for the isomerization of hydrocarbons. A corresponding use of an ionic liquid is disclosed, for example, in WO 2011/069929, where a specific selection of ionic liquids is used in the presence of an olefin for isomerization of saturated hydrocarbons, more particularly for isomerization of methylcyclopentane (MCP) to cyclohexane. A similar process is described in WO 2011/069957, but the isomerization therein is not effected in the presence of an olefin, but with a copper(II) compound.

In addition to the ionic liquid, it is also possible to use hydrogen halides, preferably as cocatalysts, in hydrocarbon conversion processes, especially in isomerization processes. Frequently, the hydrogen halides are used in gaseous form. In order to be able to better utilize the cocatalytic effect of the hydrogen halides, a partial pressure of 1-10 bar of hydrogen halide, especially of hydrogen chloride, is generally established over the reaction mixture in which the isomerization is performed. However, a certain portion of the hydrogen halide used is dissolved in the hydrocarbons and consequently discharged from the isomerization reaction. This proportion of hydrogen halide dissolved in the hydrocarbons has to be removed again from the hydrocarbons after the isomerization, particularly due to the corrosive properties of the hydrogen halide, and this removal is in practice frequently associated with problems.

US-A 2011/0155632 discloses a process for preparing products with a low hydrogen halide content, wherein the content of hydrogen halides is reduced in at least two separation stages, by stripping or distillation from a mixture which originates from a reactor and comprises an ionic liquid as a catalyst. In one embodiment of the process described in US-A 2011/0155632, the ionic liquid used as a catalyst is recycled into an alkylation reactor from a downstream phase separator, and hydrogen chloride is recycled from a first distillation column downstream of the phase separator and an isobutane-comprising stream from a second distillation column further downstream into the alkylation reactor. After the second distillation, an alkaline wash can optionally be performed in this process. US-A 2011/0155632, however, does not disclose anywhere that a hydrogen halide, especially hydrogen chloride, can be removed effectively only by means of washing with an aqueous medium having a pH between 5 and 9 from a product, for example from an alkylation product or an isomerization product. In contrast, in the execution variants described therein, the use of two separation stages by means of stripping or distillation, more particularly of two distillation stages, is absolutely necessary in order to obtain a low content of hydrogen halide in the reaction product. A similar disclosure to that in US-A 2011/0155632 is present in US-A 2011/0155640, but the process described therein relates to a hydrocarbon conversion.

U.S. Pat. No. 3,271,467 discloses a process and a corresponding apparatus for maintaining the hydrogen halide concentration in a hydrocarbon conversion, wherein the catalyst used is a metal halide and the hydrogen halide is used as a promoter. Suitable metal halides are, for example, aluminum chloride, aluminum bromide, boron trifluoride or halides of zinc, tin, antimony or zirconium, but such compounds are not ionic liquids. The hydrocarbon conversion may, for example, be an isomerization of methylcyclopentane (MCP) to cyclohexane. In a (first) stripping apparatus, a stream rich in gaseous hydrogen halide is removed from the hydrocarbon-containing output from the hydrocarbon conversion and discharged from the arrangement. A second stream enriched in hydrogen halide is passed from the stripping apparatus into an absorption apparatus, in order to selectively remove the hydrogen halide present in this stream over a solid absorber therein. The hydrogen halide thus removed is removed again from the solid absorber and recycled into the system.

WO 2010/075038 discloses a process for reducing the content of organic halides in a reaction product, these being formed as a result of a hydrocarbon conversion process in the presence of a halogen-comprising catalyst based on an acidic ionic liquid. The hydrocarbon conversion process is especially an alkylation; this process can optionally also be performed as an isomerization. The organic halides are removed from the reaction product by washing with an aqueous alkaline solution. The use of hydrogen halide as a cocatalyst of ionic liquids in hydrocarbon conversions such as in isomerization processes and the associated removal of hydrogen halide from the isomerization product, however, is not disclosed in WO 2010/075038.

It is an object of the present invention to provide a novel process for removing hydrogen halide from a mixture which is obtained in a hydrocarbon conversion, especially in an isomerization, of at least one hydrocarbon in the presence of an acidic ionic liquid.

The object is achieved by a process for treating an output from a hydrocarbon conversion, the hydrocarbon conversion being performed in the presence of an acidic ionic liquid having the composition K1AlₙX₍₃ₙ₊₁₎ where K1 is a monovalent cation, X is halogen and 1<n<2.5, the output comprising a mixture (G1) and mixture (G1) comprising at least one hydrocarbon and at least one hydrogen halide (HX), which comprises washing mixture (G1) in a wash with an aqueous medium, the aqueous medium having a pH between 5 and 9, preferably between 6 and 8, to obtain a mixture (G2) comprising at least one hydrocarbon and an amount of at least one hydrogen halide (HX) reduced by at least 90%, preferably at least 99%, compared to mixture (G1).

### A BRIEF DESCRIPTION OF THE FIGURES

FIG. 1 illustrates an example of the process according to the invention.

FIG. 2 illustrates an example of the process according to the invention.

FIG. 3 illustrates an example of the process according to the invention.

FIG. 4 illustrates an example of the process according to the invention

By virtue of the process according to the invention, it is advantageously possible to remove hydrogen halide present/dissolved in the corresponding product (hydrocarbons) after a hydrocarbon conversion, especially an isomerization, from this product again, especially from an isomerization product.

The advantages are considered to be primarily economic, because, with respect to processes in which hydrogen halides are removed fully or partly with an alkaline wash, the additional costs which are caused by the use of alkaline additions, especially of sodium hydroxide, can be eliminated. In addition, such (strongly) alkaline additions are associated with an elevated level of apparatus complexity. The economic advantages and/or apparatus simplification of the process according to the invention are manifested particularly when the inventive wash with the aqueous medium is performed in multiple stages and in countercurrent mode and/or using at least one dispersion and phase separation unit, especially a mixer-settler apparatus, per wash stage or at least one extraction column. The aforementioned advantages are manifested especially over the processes described in US-A 2011/0155632 or US-A 2011/0155640 for removal of hydrogen halides.

The above-described advantages of the process according to the invention become even more apparent if the inventive wash step is preceded by upstream connection of a phase separation unit, especially a phase separator, and/or an apparatus (V1), especially a flash apparatus or a stripping apparatus, for gaseous preliminary removal of a portion of hydrogen halide.

If the apparatus (V1) used in the process according to the invention is a flash apparatus, this is associated with further advantages. The use of a flash apparatus in the optional step a) is first of all less costly and simpler in apparatus terms, especially compared to the use of a rectifying column (due to the corrosiveness of the hydrogen halide, which is particularly disadvantageous given the complex geometries which exist in a column). The separation effect in the flash apparatus is advantageously achieved merely by lowering the pressure relative to the pressure selected for the hydrocarbon conversion, especially for the isomerization. Thus, no separate energy input is needed, and the corrosiveness of the hydrogen halide is less apparent as a result.

The process according to the invention for treatment of an output from a hydrocarbon conversion, wherein the hydrocarbon conversion is performed in the presence of an acidic ionic liquid, is defined in detail hereinafter.

Hydrocarbon conversions as such are known to those skilled in the art. The hydrocarbon conversion is preferably selected from an alkylation, a polymerization, a dimerization, an oligomerization, an acylation, a metathesis, a polymerization or copolymerization, an isomerization, a carbonylation or combinations thereof. Alkylations, isomerizations, polymerizations etc. are known to those skilled in the art. Especially preferably in the context of the present invention, the hydrocarbon conversion is an isomerization.

In the context of the present invention, the hydrocarbon conversion is effected in the presence of an acidic ionic liquid having the composition K1AlₙX₍₃ₙ₊₁₎ where K1 is a monovalent cation, X is halogen and 1<n<2.5. Such acidic ionic liquids are known to those skilled in the art; they are disclosed (alongside further ionic liquids), for example, in WO 2011/069929. For example, mixtures of two or more acidic ionic liquids may be used, preference being given to using one acidic ionic liquid.

K1 is preferably an unsubstituted or at least partly alkylated ammonium ion or a heterocyclic (monovalent) cation, especially a pyridinium ion, an imidazolium ion, a pyridazinium ion, a pyrazolium ion, an imidazolinium ion, a thiazolium ion, a triazolium ion, a pyrrolidinium ion, an imidazolidinium ion or a phosphonium ion. X is preferably chlorine or bromine.

The acidic ionic liquid more preferably comprises, as a cation, an at least partly alkylated ammonium ion or a heterocyclic cation and/or, as an anion, a chloroaluminate ion having the composition AlₙCl₍₃ₙ₊₁₎ where 1<n<2.5. The at least partly alkylated ammonium ion preferably comprises one, two or three alkyl radicals (each) having 1 to 10 carbon atoms. If two or three alkyl substituents are present with the corresponding ammonium ions, the respective chain length can be selected independently; preferably, all alkyl substituents have the same chain length. Particular preference is given to trialkylated ammonium ions having a chain length of 1 to 3 carbon atoms. The heterocyclic cation is preferably an imidazolium ion or a pyridinium ion.

The acidic ionic liquid especially preferably comprises, as a cation, an at least partly alkylated ammonium ion and, as an anion, a chloroaluminate ion having the composition AlₙCl₍₃ₙ₊₁₎ where 1<n<2.5. Examples of such particularly preferred acidic ionic liquids are trimethylammonium chloroaluminate and triethylammonium chloroaluminate.

The acidic ionic liquid used in the context of the present invention is preferably used as a catalyst in the hydrocarbon conversion, especially as an isomerization catalyst. In addition, in the context of the present invention, the hydrocarbon conversion is also effected in the presence of a hydrogen halide (HX), preference being given to using the hydrogen halide (HX) as a cocatalyst.

The hydrogen halides (HX) used may in principle be any conceivable hydrogen halides, for example hydrogen fluoride (HF), hydrogen chloride (HCl), hydrogen bromide (HBr) or hydrogen iodide (HI). The hydrogen halides can optionally also be used as a mixture, but preference is given in the context of the present invention to using only one hydrogen halide. Preference is given to using the hydrogen halide whose halide moiety is also present in the above-described acidic ionic liquid (at least partly) in the corresponding anion. The hydrogen halide (HX) is preferably hydrogen chloride (HCl) or hydrogen bromide (HBr). The hydrogen halide (HX) is more preferably hydrogen chloride (HCl).

In principle, it is possible in the context of the present invention to use any hydrocarbons, provided that at least one of the hydrocarbons used can be subjected in the presence of the above-described acidic ionic liquids to a hydrocarbon conversion, especially to an isomerization. On the basis of his or her specialist knowledge, the person skilled in the art knows which hydrocarbons can be subjected by means of acidic ionic liquids to a hydrocarbon conversion, and more particularly which hydrocarbons are isomerizable. For example, it is possible to use mixtures of two or more hydrocarbons, but it is also possible to use only one hydrocarbon. Thus, it is possible in the context of the present invention that, in a mixture comprising two or more hydrocarbons, only one of these hydrocarbons is subjected to a hydrocarbon conversion, especially isomerized. Optionally, such mixtures may also comprise compounds which are not themselves hydrocarbons but are miscible therewith.

The hydrocarbon used in the hydrocarbon conversion is preferably methylcyclopentane (MCP) or a mixture of methylcyclopentane (MCP) with at least one further hydrocarbon selected from cyclohexane, n-hexane, isohexanes, n-heptane, isoheptanes, methylcyclohexane or dimethylcyclopentanes.

More preferably, a mixture of methylcyclopentane (MCP) with at least one further hydrocarbon selected from cyclohexane, n-hexane, isohexanes, n-heptane, isoheptanes, methylcyclohexane or dimethylcyclopentanes is used.

The hydrocarbon conversion can in principle be performed in all apparatuses known for such a purpose to the person skilled in the art. The corresponding apparatus is preferably a stirred tank or a stirred tank cascade. A “stirred tank cascade” means that two or more, for example three or four, stirred tanks are connected in succession (in series).

As already explained above, due to the hydrocarbon conversion in the presence of an acidic ionic liquid and of a hydrogen halide (HX), the chemical structure of at least one of the hydrocarbons used is altered. The hydrocarbons obtained in the hydrocarbon conversion are present in a mixture (G1). Mixture (G1) thus differs in terms of (chemical) composition and/or amount of the hydrocarbons present therein from the corresponding hydrocarbon composition present prior to the hydrocarbon conversion, especially prior to the isomerization. Since the hydrocarbon conversion to be performed in such hydrocarbon conversions, especially in isomerization processes, frequently does not proceed to an extent of 100% (i.e. to completion), the product generally still also comprises the hydrocarbon with which the hydrocarbon conversion has been performed (in a smaller amount than before the hydrocarbon conversion). If, for example, MCP is to be isomerized to cyclohexane, the isomerization product frequently comprises a mixture of cyclohexane and (in a smaller amount than before the isomerization) MCP.

As well as the hydrocarbons, mixture (G1) comprises at least one hydrogen halide (HX) and optionally further components. The hydrogen halide (HX) present in mixture (G1) is generally the same hydrogen halide as that used in the hydrocarbon conversion (preferably as a cocatalyst), because the chemical structure of the hydrogen halide is not normally altered by the hydrocarbon conversion, but there may be partial exchange of the anionic moiety of the hydrogen halide used with other halide ions present in the process. As a further component, mixture (G1) preferably comprises the above-described ionic liquid. Mixture (G1) additionally comprises between 10 and 99% by weight, preferably between 50 and 95% by weight, of acidic ionic liquid (the stated amounts are based on the total weight of hydrocarbons and hydrogen halide in mixture (G1)).

The hydrocarbon present in mixture (G1)—i.e. as the product of the hydrocarbon conversion—is preferably cyclohexane. The hydrocarbon present in mixture (G1) is more preferably cyclohexane or a mixture of cyclohexane with at least one further hydrocarbon selected from methylcyclopentane (MCP), n-hexane, isohexanes, n-heptane, isoheptanes, methylcyclohexane and dimethylcyclopentane.

The hydrocarbon present in mixture (G1) is especially preferably a mixture of cyclohexane, MCP and at least one further hydrocarbon. The further hydrocarbon is preferably selected from n-hexane, isohexanes, n-heptane, isoheptanes, methylcyclohexane and dimethylcyclopentanes. If the hydrocarbon conversion is performed as an isomerization, the proportion of branched hydrocarbons in mixture (G1) is preferably less than 10% by weight (based on the sum of all hydrocarbons present in mixture (G1)). Particular preference is given in the context of the present invention to isomerizing methylcyclopentane (MCP) to cyclohexane.

In a preferred embodiment of the present invention, mixture (G1) comprises i) as a hydrocarbon a mixture of cyclohexane with at least one further hydrocarbon selected from methylcyclopentane (MCP), n-hexane, isohexanes, n-heptane, isoheptanes, methylcyclohexane and dimethylcyclopentanes, ii) hydrogen chloride (HCl) and iii) an acidic ionic liquid which has, as a cation, an at least partly alkylated ammonium ion and, as an anion, a chloroaluminate ion having the composition AlₙCl₍₃ₙ₊₁₎ where 1<n<2.5.

In a further preferred embodiment of the present invention, the hydrocarbons present in mixture (G1), to an extent of at least 80% by weight, have at least 5 carbon atoms per molecule. These hydrocarbons especially preferably have at least 6 carbon atoms per molecule.

Mixture (G1) is at first present in the apparatus in which the hydrocarbon conversion is performed. In the context of the process according to the invention, mixture (G1) is discharged from this apparatus as the output. In other words, this means that the output comprises mixture (G1) and the output or mixture (G1), after it has left the apparatus for performance of the hydrocarbon conversion, is subjected to the inventive wash with an aqueous medium having a pH between 5 and 9 (wash step). The inventive wash can be performed in all apparatuses known for this purpose to those skilled in the art.

According to the invention, this wash step is performed in such a way that mixture (G1) is washed in a wash with an aqueous medium, the aqueous medium having a pH (pH value) between 5 and 9. The pH preferably has a value between 6 and 8. As a result of this wash step, a mixture (G2) is obtained which comprises at least one hydrocarbon and an amount of at least one hydrogen halide (HX) reduced by at least 90% compared to mixture (G1). The amount of at least one hydrogen halide is preferably reduced by at least 99%.

The aqueous medium is preferably water, especially preferably demineralized water. It is additionally preferred that the aqueous medium is substantially free or completely free of alkali metal hydroxides, especially of NaOH. The expression “substantially free of alkali metal hydroxides” in the context of the present invention is understood to mean that, in the corresponding aqueous medium, at most small amounts of such ofalkali metal hydroxides are still present, the upper limit in the amount of alkali metal hydroxides still tolerable being laid down by the maximum possible pH of 9. The upper limit of alkali metal hydroxides is preferably 100 ppm (based on the total weight of the aqueous medium).

In a preferred embodiment of the present invention, the wash (wash step) is performed in multiple stages, preferably at least 3 stages. It is additionally preferred that the multistage wash of mixture (G1) with the aqueous medium is performed in countercurrent mode.

In one embodiment of the present invention, only a one-stage wash step is performed, in which case the aqueous medium has a pH of 5 to 9, preferably between 6 and 8, and is especially preferably demineralized water.

In addition, it is preferable in the context of the present invention that the one-stage or multistage wash, preferably the multistage wash, is performed using at least one dispersion and phase separation unit or at least one extraction column per wash stage. The dispersion and phase separation unit is preferably a mixer-settler apparatus (combination of a stirred tank with a downstream phase separator), a combination of at least one static mixer with at least one phase separator or a combination of at least one mixing pump with at least one phase separator.

In another embodiment, the wash step is performed in a multistage mixer-settler apparatus, preferably operated in countercurrent, or extraction is effected with water in an extraction column operated in countercurrent. In the case of the mixer-settler apparatus or extraction column, a further wash stage is preferably connected downstream thereof in flow direction of mixture (G1) (comprising the hydrocarbons), this being fed with fresh water. In the aqueous outlet thereof is an apparatus for continuous measurement of the pH or the electrical conductivity, in order thus to monitor the complete removal of the non-hydrocarbon components, especially HCl.

In the context of the process according to the invention, it is preferable that mixture (G2) obtained in the wash step, with regard to the composition and/or amount of the hydrocarbons present therein, corresponds completely or at least substantially to mixture (G1). The expression “corresponds substantially” shall be understood in this context to mean that at least 90% by weight, preferably at least 95% by weight, especially at least 99% by weight, of the hydrocarbons present in mixture (G1) are also present in mixture (G3). Especially preferably, mixture (G3) does not comprise any further components apart from at least one hydrocarbon and not more than 100 ppm by weight, preferably not more than 10 ppm by weight, of hydrogen halide. The same also applies to the embodiments of the present invention described in the text below in which, rather than mixture (G1), due to optional intermediate steps, mixtures such as (G1*) or (G1)-IL are subjected to the inventive wash.

If the hydrocarbon conversion in the context of the present invention is an isomerization, the isomerization is preferably performed as follows. The performance of an isomerization of hydrocarbons in the presence of an ionic liquid as a catalyst and a hydrogen halide as a cocatalyst is known to those skilled in the art. The hydrocarbons and the ionic liquid in the isomerization preferably each form a separate phase, though portions of the ionic liquid may be present in the hydrocarbon phase and portions of the hydrocarbons in the ionic liquid phase. The hydrogen halide, especially hydrogen chloride, is introduced, preferably in gaseous form, into the apparatus for performance of the isomerization. The hydrogen halide may be present, at least in portions, in the two aforementioned liquid phases; the hydrogen halide preferably forms a separate, gaseous phase.

The isomerization is preferably performed at a temperature between 0° C. and 100° C., especially preferably at a temperature between 30° C. and 60° C. It is additionally preferred that the pressure in the isomerization is between 1 and 20 bar abs. (absolute), preferably between 2 and 10 bar abs.

The isomerization is preferably performed in the apparatus in such a way that two liquid phases and one gaseous phase are present in a stirred tank or a stirred tank cascade. The first liquid phase comprises the acidic ionic liquid to an extent of at least 90% by weight and the second liquid phase comprises the hydrocarbons to an extent of at least 90% by weight. The gas phase comprises at least one hydrogen halide, preferably hydrogen chloride, to an extent of at least 90% by weight. Optionally, a solid phase may also be present, this comprising components from which the ionic liquid is formed in solid form, for example AlCl₃. The pressure and composition of the gas phase are set here such that the partial pressure of the gaseous hydrogen halide, especially of HCl gas, in the gas phase is between 1 and 20 bar abs., preferably between 2 and 10 bar abs.

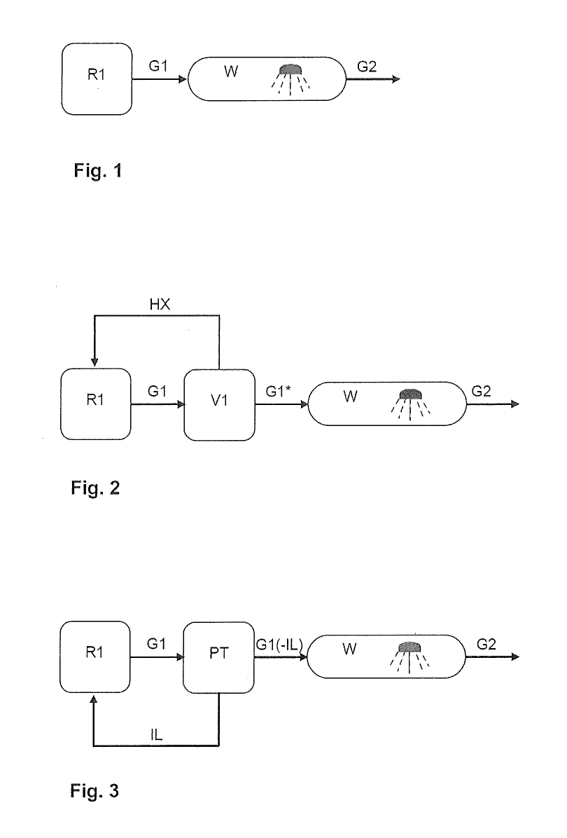

FIG. 1 once again illustrates the process according to the invention. R1 represents the apparatus in which the hydrocarbon conversion, especially an isomerization, is performed. This is preferably a stirred tank or a stirred tank cascade. According to FIG. 1, the mixture (G1) discharged from R1 is washed with the aqueous medium without any intermediate steps. In FIG. 1, the inventive wash step is referred to in simplified form with the abbreviation “W”. The wash step according to FIG. 1 may, as described above, be performed in one or more stages, in which case preference is given to performing a multistage wash of mixture (G1) in countercurrent to the aqueous medium, and/or a dispersion and phase separation unit, especially a mixer-settler apparatus or an extraction column, is used.

In a preferred embodiment of the present invention, the wash of mixture (G1) is preceded by performance of the following steps (a and b):
a) feeding mixture (G1) into an apparatus (V1), and drawing off at least 50%, preferably at least 70%, of the hydrogen halide (HX) present in (G1) in gaseous form from (V1),
b) discharging a mixture (G1*) from apparatus (V1), mixture (G1*) comprising at least one hydrocarbon and an amount of at least one hydrogen halide (HX) reduced by the gaseous stream according to step a) compared to mixture (G1), and the subsequent wash being performed with mixture (G1*) rather than mixture (G1).

The apparatus (V1) used to perform the gaseous drawing-off (removal) of the hydrogen halide (HX) from mixture (G1) according to step a) may in principle be any apparatus known for such a purpose to the person skilled in the art, preferably a concentration apparatus, a rectifying column, an apparatus for flash vaporization (flash apparatus) or a stripping apparatus. V1 is especially preferably a flash apparatus.

Apparatus (V1) is intended, in the context of the process according to the invention, preferably to perform a preliminary removal of the hydrogen halides from the hydrocarbons, and then, in the subsequent inventive wash step, to remove the amount of hydrogen halides still remaining from the hydrocarbons.

In the context of the present invention, step a) should be understood such that, in the case of use of a flash apparatus as apparatus (V1), an appropriate flash operation (flashing) is performed with mixture (G1). The same applies to the further configurations of apparatus (V1) detailed above, such as stripping apparatus or vaporizer.

In the context of the present invention, the term “concentration”, which is performed in a corresponding concentration apparatus, is understood to mean the following: a characteristic feature of concentration is that a portion of the liquid mixture to be separated is vaporized with supply of heat and is condensed after removal from the remaining liquid mixture. For the original liquid phase, a vapor phase is thus produced, in which the relatively low-boiling mixture components are enriched.

In the context of the present invention, the term “rectification”, which is performed in a corresponding rectifying column (rectifying apparatus), also called rectification column (rectification apparatus), is understood to mean the following: in rectification, the vapor produced by distillation is conducted in countercurrent to a portion of the condensate thereof in a rectifying column. In this way, more volatile components are enriched in the top product and less volatile components in the bottom product of the rectification column.

In the context of the present invention, the term “flashing”, which is performed in a corresponding flash apparatus and can also be referred to as flash vaporization, is understood to mean the following: Flash vaporization (flashing) involves decompressing a liquid mixture into a suitable apparatus (flash apparatus), for example into a vapor/liquid separation vessel (i.e., in a suitable apparatus, for example a valve, a lowering of the pressure finds off, this being sufficient to cause a portion of the liquid mixture to vaporize spontaneously). The liquid mixture may originate, for example, from a reaction stage operated at higher pressure. However, it is also possible to effect preheating in a preheater, for example to boiling temperature, in which case the pressure in the preheater must be higher than the pressure in the downstream separation vessel. The vapor forming in the course of decompression has a higher proportion of relatively low-boiling components than the mixture entering the separator. The flash evaporation thus ensures partial separation of the incoming mixture, in which case the separator can act as a sole theoretical plate. The flashing can also be combined with heat supply to the liquid mixture which remains in the flashing operation, for example by means of a circulation vaporizer connected to the separation vessel.

In the context of the present invention, the term “stripping”, which is performed in a corresponding stripping apparatus, is understood to mean the following: in the course of stripping, one or more relatively low-boiling components are depleted from a liquid, these being contacted, preferably in a countercurrent column, with gases such as nitrogen, air or steam, such that the decrease in the partial pressure of the relatively low-boiling components in the gas phase brought about by the gas results in a decrease in the solubility thereof in the liquid.

Further information regarding the above terms “distillation”, “rectification”, “vaporization”, “flashing” and/or “stripping” can be found in the following textbooks: Sattler, Thermische Trennverfahren [Thermal Separation Processes], VCH, 1988; Perry's Chemical Engineers' Handbook, 7th edition; R. H. Perry, D. W. Green, 1997, McGraw-H ill.

In a further preferred embodiment of the present invention, in step a), hydrogen halide (HX) drawn off via mixture (G1b) is recycled fully or partly by means of a suitable apparatus, for example a jet compressor, piston compressor, turbo compressor or screw compressor, into the apparatus in which the hydrocarbon conversion, especially the isomerization, is performed. If complete recycling of mixture (G1b) is not performed, the excess amounts of mixture (G1b) are discharged from the process according to the invention and (generally) discarded or sent to a further process step.

In the optional additional step b), mixture (G1*) preferably comprises an amount of at least one hydrogen halide (HX) reduced by at least 50%. More preferably, mixture (G1*) comprises an amount of at least one hydrogen halide (HX) reduced relative to mixture (G1) by at least 70%.

Preference is given to performing the optional step b) according to at least one, more preferably according to both, of the following variants i) and ii):
i) at least 95% by weight of mixture (G1*) discharged from apparatus (V1) is liquid,
iii) the discharged mixture (G1*) is at most 150 K, preferably at most 100 K, hotter than the mixture (G1b) drawn off (according to step a)).

In a particularly preferred embodiment of the present invention, a one-stage vaporization, especially a one-stage flash vaporization, takes place in apparatus (V1) and the mixture (G1*) discharged from apparatus (V1) is washed with the aqueous medium without any intermediate steps.

FIG. 2 once again illustrates the process according to the invention in a preferred embodiment. R1 represents the apparatus in which the hydrocarbon conversion, especially an isomerization, is performed. This is preferably a stirred tank or a stirred tank cascade. Apparatus (V1) is preferably a vaporizer, especially a flash apparatus. In this embodiment, recycling of the mixture (G1b) removed, preferably in gaseous form, from apparatus (V1) is also performed. According to FIG. 2, the mixture (G1*) discharged from apparatus (V1) is washed with the aqueous medium without any intermediate steps. For the rest, in FIG. 2, the abbreviations, arrows and other symbols have similar meaning to those explained above for FIG. 1.

In a further preferred embodiment of the present invention, mixture (G1) is discharged as an output from the apparatus in which the hydrocarbon conversion is performed, conducted through a phase separation unit, especially into a phase separator, and then washed in the inventive wash. In other words, this means that, after the performance of the hydrocarbon conversion and prior to performance of the inventive wash step, an alternative intermediate step is performed using an aqueous medium (with respect to the embodiment illustrated in FIG. 2). In this intermediate step, the acidic ionic liquid present in mixture (G1) is fully or at least partly removed from mixture (G1), and then mixture (G1)-IL depleted of acidic ionic liquid is subjected to a wash with an aqueous medium having a pH between 5 and 9. This intermediate step, in contrast to the above-described embodiments with inclusion of the likewise optional steps a) and b), serves primarily to remove not hydrogen halide from mixture (G1), but the ionic liquid preferably likewise present in mixture (G1). Optionally, however, portions of the hydrogen halides present in mixture (G1) may also be removed from mixture (G1) in this intermediate step together with the ionic liquid.

Preferably at least 90%, more preferably at least 99%, of the acidic ionic liquid is removed from mixture (G1) in the phase separation unit and optionally recycled partly or fully into the apparatus in which the hydrocarbon conversion is performed.

The above-described further preferred embodiment of the present invention is additionally illustrated in FIG. 3. In FIG. 3, the abbreviations, arrows and other symbols have similar meanings to those explained above for FIGS. 1 and 2; PT means phase separation unit, IL means acidic ionic liquid.

In a further preferred embodiment of the present invention, mixture (G1) is discharged as an output from the apparatus in which the hydrocarbon conversion is performed, conducted through a phase separation unit, especially into a phase separator, and then fed into the apparatus (V1) for performance of steps a) and b). In other words, this means that, after the performance of the hydrocarbon conversion and prior to performance of the inventive wash step with the aqueous medium, two intermediate steps (removal of ionic liquid in the phase separation unit and subsequent removal of hydrogen halide in apparatus (V1)) are performed. This embodiment thus constitutes a combination of the above-described embodiments which have been illustrated in FIGS. 2 and 3.

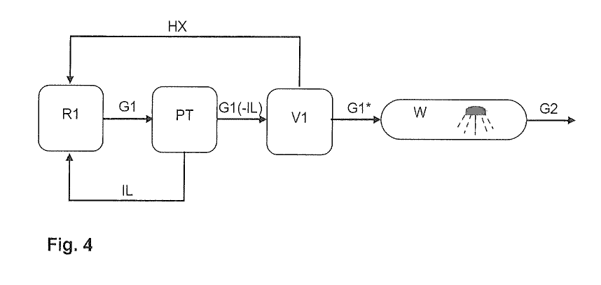

The above-described further preferred embodiment of the present invention is additionally illustrated in FIG. 4. In FIG. 4, the abbreviations, arrows and other symbols have similar meanings to those explained above for FIGS. 1 to 3.

In the context of the present invention, cyclohexane is preferably isolated from mixture (G2). Processes and apparatuses for removal of cyclohexane from mixture (G2) are known to those skilled in the art.

The present invention is to be illustrated hereinafter by examples.

The output (G1) from the hydrocarbon conversion, in which methylcyclopentane is isomerized to cyclohexane at 50° C. in the presence of the superacidic ionic liquid trimethylammonium heptachlorodialuminate, comprises, as organic constituents (“organics”), cyclohexane (58%), methylcyclopentane (13%), n-hexane (22%) and isohexanes (7%). This corresponding output is decompressed from gauge pressure 2 bar to standard pressure in a flash apparatus (VI). In the course of this, the majority of the HCl dissolved in the organics escapes in gaseous form (90%).

The corresponding halide content (HCl) of this output after decompression (G1*) and after washing once, twice and three times with the same amount of demineralized water (pH of 6.1) is subsequently determined. The procedure here is as follows:

The respective organic phase obtained (G1* or G2 after washing once (1), twice (2) and three times (3)) is admixed with 10 ml of NaOH (0.1 molar) and the excess sodium hydroxide solution is then subsequently back-titrated with concentrated H₂SO₄. Thus, the corresponding halide content of the individual phases can be determined.

The results of the HCl determination of the output G1* and after the individual wash steps are collated in table 1.

TABLE 1
Results of the HCl determination of the individual phases after
decompression in the flash apparatus (G1*) and the respective
wash steps with demineralized water. The 1840 ppm of HCl
corresponds here to the amount of HCl dissolved in the organics
after decompression to standard pressure. By washing with
water, this content of HCl can be significantly reduced further.

| | | | HCl content in the |
|---|---|---|---|
| | Organics used | Amount of water | corresponding |
| Phase | [ml] | [ml] | phase [ppm] |
| G1* | 100 | | 1840 |
| G2 (1) | 60 | 60 | 110 |
| G2 (2) | 52 | 52 | 70 |
| G2 (3) | 41 | 41 | 20 |

As can be seen from the analysis values in table 1, the chlorine content in the organic output can be reduced by 99% compared to the organic phase after decompression by washing three times with demineralized water.

In the case of a corresponding alkaline wash with 4% sodium hydroxide solution, about 50 ml is needed to neutralize 1 liter of the corresponding organics. This relatively costly alkaline wash can thus be replaced by a neutral wash with demineralised water, in order thus correspondingly to reduce the costs.

1.-20. (canceled)

21. A process for treating an output from a hydrocarbon conversion, the hydrocarbon conversion being performed in the presence of an acidic ionic liquid having the composition K1AlₙX₍₃ₙ₊₁₎ where K1 is a monovalent cation, X is halogen and 1<n<2.5, the output comprising a mixture (G1) and mixture (G1) comprising at least one hydrocarbon and at least one hydrogen halide (HX), which comprises washing mixture (G1) in a wash with an aqueous medium, the aqueous medium having a pH between 5 and 9 to obtain a mixture (G2) comprising at least one hydrocarbon and an amount of at least one hydrogen halide (HX) reduced by at least 90% compared to mixture (G1).

22. The process according to claim 21, wherein the aqueous medium has a pH between 6 and 8 or the amount of at least one hydrogen halide (HX) is reduced by at least 99%.

23. The process according to claim 21, wherein the aqueous medium is demineralized water.

24. The process according to claim 21, wherein the wash is performed in multiple stages.

25. The process according to claim 24, wherein multiple stage is at least 3 stages.

26. The process according to claim 24, wherein the multistage wash of mixture (G1) with the aqueous medium is performed in countercurrent mode.

27. The process according to claim 21, wherein the wash of mixture (G1) is preceded by performance of the following steps:
a) feeding mixture (G1) into an apparatus (V1), and drawing off at least 50% of the hydrogen halide (HX) present in (G1) in gaseous form from (V1),
b) discharging a mixture (G1*) from apparatus (V1), mixture (G1*) comprising at least one hydrocarbon and an amount of at least one hydrogen halide (HX) reduced by the gaseous stream according to step a) compared to mixture (G1), and the subsequent wash being performed with mixture (G1*) rather than mixture (G1).

28. The process according to claim 21, wherein the hydrocarbon conversion is selected from an alkylation, a polymerization, a dimerization, an oligomerization, an acylation, a metathesis, a polymerization or copolymerization, an isomerization, a carbonylation or combinations thereof.

29. The process according to claim 28, wherein the hydrocarbon conversion is an isomerization, of methylcyclopentane (MCP) to cyclohexane.

30. The process according to claim 229, wherein the apparatus (V1) is a concentration apparatus, a rectifying column, a flash apparatus or a stripping apparatus.

31. The process according to claim 21, wherein the hydrogen halide (HX) is hydrogen chloride (HCl).

32. The process according to claim 27, wherein the mixture (G1*) discharged from apparatus (V1) is washed with the aqueous medium without any intermediate steps.

33. The process according to claim 27, wherein hydrogen halide (HX) drawn off in step a) is recycled into the apparatus in which the hydrocarbon conversion, is performed.

34. The process according to claim 21, wherein mixture (G1) comprises, as the hydrocarbon, cyclohexane or a mixture of cyclohexane with at least one further hydrocarbon selected from methylcyclopentane (MCP), n-hexane, isohexanes, n-heptane, isoheptanes, methylcyclohexane or dimethylcyclopentanes.

35. The process according to claim 21, wherein the acidic ionic liquid comprises, as a cation, an at least partly alkylated ammonium ion or a heterocyclic cation or, as an anion, a chloroaluminate ion having the composition AlₙCl₍₃ₙ₊₁₎ where 1<n<2.5.

36. The process according to claim 21, wherein mixture (G1) additionally comprises between 10 and 99% by weight of acidic ionic liquid.

37. The process according to claim 21, wherein mixture (G1) is discharged as an output from the apparatus in which the hydrocarbon conversion is performed, conducted through a phase separation unit and then washed in the wash.

38. The process according to claim 37, wherein the phase separation unit is a phase separator.

39. The process according to claim 37, wherein at least 90% of the acidic ionic liquid is removed from mixture (G1) in the phase separation unit and optionally recycled into the apparatus in which the hydrocarbon conversion is performed.

40. The process according to claim 37, wherein mixture (G1), after it is conducted through the phase separation unit and before it is washed in the wash, is used to perform process steps a) and b).

41. The process according to claim 21, wherein cyclohexane is isolated from mixture (G2).

42. The process according to any claim 21, wherein the one-stage or multistage wash is performed using at least one dispersion and phase separation unit per wash stage or at least one extraction column.

43. The process according to claim 42, wherein the dispersion and phase separation unit is a mixer-settler apparatus, a combination of at least one static mixer with at least one phase separator or a combination of at least one mixing pump with at least one phase separator.
